(19) United States
(12) Patent Application Publication
(10) Pub. No.: US 2014/0074407 A1
(43) Pub. Date: Mar. 13, 2014

(54) DEVICE AND METHOD FOR ESTIMATING ENERGY EXPENDITURE DURING EXERCISE

(71) Applicant: TOUMAZ HEALTHCARE LIMITED, Oxfordshire (GB)

(72) Inventors: Miguel HERNANDEZ-SILVEIRA, Oxfordshire (GB); Su-Shin ANG, Oxfordshire (GB)

(73) Assignee: TOUMAZ HEALTHCARE LIMITED, Oxfordshire (GB)

(21) Appl. No.: 14/021,015

(22) Filed: Sep. 9, 2013

(30) Foreign Application Priority Data
Sep. 7, 2012 (GB) 1216014.9

(51) Int. Cl. A61B 5/11

(52) U.S. Cl. CPC A61B 5/1118
USPC 702/19; 600/529; 600/509; 600/521

(57) ABSTRACT

The present invention relates to a device and method for estimating energy expenditure during exercise. The device includes a module for estimating whether a person is exceeding their anaerobic threshold and, if they are exceeding their anaerobic threshold calculating the additional energy expenditure due to the anaerobic metabolism of ATP. The additional energy expenditure can then be added to an estimate of the energy expenditure due to aerobic metabolism and output to the user in order to provide an estimate of the energy expenditure occurring during anaerobic exercise.

### CROSS-REFERENCE TO RELATED APPLICATIONS

This application is based on, and claims priority to, Great Britain Application No. 1216014.9, filed Sep. 7, 2012, the entire contents of which is hereby incorporated fully by reference.

### FIELD OF THE INVENTION

The present invention relates to a device and method for estimating energy expenditure during exercise. It is of particular use in providing a more accurate estimate of energy expenditure when exercising above the anaerobic threshold.

### BACKGROUND OF THE INVENTION

There are three sub-systems in the human body that are responsible for the release of energy: aerobic metabolism, alactic anaerobic metabolism (phosphagen system), and anaerobic lactic metabolism. In all of these systems, energy release is achieved through the breakdown of Adenosine Triphosphate (ATP) into Adenosine Diphosphate (ADP). These systems differ in the type of biochemical reactions that are used in the production of ATP, and the efficiency of ATP production.

When the physical workload placed on a subject is gradually increased from rest to strenuous activities, ATP stored within the muscles is utilized for the initial release of energy by means of alactic anaerobic metabolism. Small amounts of energy are produced by anaerobic lactic metabolism, with lactic acid (lactate) as the by-product. However, the lactate produced by alactic metabolism is buffered by bicarbonate in the bloodstream, leading to an increased rate of carbon dioxide production, without a reduction in the blood pH. The rate of oxygen uptake increases proportionally with the rate of carbon dioxide production.

Upon depletion of the ATP stored within the muscles, which occurs after a very short period of time, aerobic metabolism becomes the dominant source of energy release. In aerobic metabolism glucose is efficiently converted into ATP for further energy release.

When the workload is increased further there is insufficient oxygen in the blood to support aerobic metabolism and, therefore, anaerobic lactic metabolism overtakes aerobic metabolism as the dominant system for producing ATP. Anaerobic lactic metabolism is relatively inefficient at producing ATP molecules from glucose, producing 2 molecules of ATP for every molecule of glucose compared to aerobic metabolism producing 38 molecules of ATP for every molecule of glucose. Additionally, the lactic acid produced by the anaerobic respiration cannot be completely buffered within the blood leading to an increased rate of carbon dioxide release relative to oxygen uptake by the body. The build-up of lactate in the blood eventually leads to fatigue and breathlessness. The point at which lactate begins to build in a person's blood is known as the anaerobic threshold.

The efficiency of the energy release in the body can therefore be seen to differ depending on whether a subject is carrying out predominantly aerobic metabolism (below the anaerobic threshold) or predominantly anaerobic metabolism (above the anaerobic threshold) due to differences in efficiencies between the different energy sub-systems.

For example, compared to a highly trained athlete, a typically sedentary individual is likely to exceed their anaerobic threshold earlier and at lighter workloads.

Consequently, even for the same workload, the energy expenditure by a sedentary individual will be greater than that of a highly trained athlete. This is because energy expenditure beyond the anaerobic threshold is greater than the energy expenditure below the anaerobic threshold due to the additional stress placed on the cardiovascular and respiratory systems for carbon dioxide removal.

U.S. Pat. No. 6,554,776 describes a cardiopulmonary weight-loss system that computes the energy expenditure as a linear function of oxygen uptake and carbon dioxide production, as well as the point at which the anaerobic threshold has been exceeded. This aim of the system is to enhance weight-loss by maximizing fat burning. Fat burning is maximal during aerobic respiration and, thus, the system encourages users to stay beneath the anaerobic threshold and only calculates energy expenditure using an aerobic respiration model.

U.S. Pat. No. 7,470,234 describes a portable device that monitors the vital signs of the subject, including the calorie expenditure, the real-time heart rate, and the anaerobic threshold to allow an individual to track their exercise. Energy expenditure is estimated by means of the intensity of the exercise and is calculated using an aerobic metabolism model. The anaerobic threshold is provided to a user to enable them to train within their anaerobic threshold zone.

U.S. Pat. No. 7,648,463 describes eyewear that makes use of an optical sensor to detect the flow of blood, and correspondingly calculate the heart rate of the subject. The energy expenditure is then determined as a function of the heart rate assuming that the user is using aerobic metabolism.

### SUMMARY OF THE INVENTION

According to an aspect of the present invention there is provided a device comprising an input configured to receive data from a sensor, an aerobic processor configured to estimate energy expenditure from the data using an aerobic metabolism model, an anaerobic threshold module configured to determine from the data whether the anaerobic threshold for the user has been exceeded, an output to output the estimated physical activity intensity if the anaerobic threshold for the user has not been exceeded, an anaerobic energy expenditure estimator configured to estimate energy expenditure due to anaerobic respiration from the data, a processor configured to combine the estimated anaerobic energy expenditure and the estimated aerobic energy expenditure to produce a total energy expenditure when the anaerobic threshold for the user has been exceeded, and an output to output the total energy expenditure if the anaerobic threshold for the user has been exceeded. By applying different models to calculate energy expenditure depending on whether the user is operating above or below their anaerobic threshold the user can be provided with a more accurate estimate of their energy expenditure.

The aerobic processor may include an exercise intensity classifier configured to classify the exercise of the user using data received by the input; and an estimator to estimate physical activity intensity and the estimated oxygen level in the respiratory gases according to the classification of the exercise wherein the output outputs the estimated physical activity intensity if the anaerobic threshold for the user has not been exceeded. By classifying the intensity of the exercise prior to estimating the physical activity intensity the most appropriate model for calculating energy expenditure due to aerobic respiration may be selected.

The anaerobic energy expenditure estimator may include a lactate estimator to estimate plasma lactate concentration; and a lactate to calorie convertor to convert the estimated plasma lactate concentration to a calorie value representing the anaerobic energy. The device may further comprise an aerobic energy expenditure estimator configured to calculate a value representing the aerobic energy expenditure using the estimated oxygen level in the respiratory gases and a processor configured to add the calorie value representing the anaerobic energy and the calorie value representing the aerobic energy expenditure to provide an estimate of the total energy expenditure when the anaerobic threshold has been passed.

The plasma lactate concentration may be estimated using an existing method:

LA=e^{(0.082logVO}^{₂}^{-0.2)} where VO₂<1.51

LA=e^{(2.88logVO}^{₂}^{-0.7)} where VO₂≧1.51

where LA is the plasma lactate concentration.

The estimated plasma lactate concentration may be converted to a calorie value representing the energy used during anaerobic respiration using the equation:

$${PAI}_{anaerobic} = \frac{20 LA}{0.75}$$

The anaerobic energy expenditure estimator calculates the calories using the following equation:

PAI_{aerobic}=5.01*VO₂

The sensor may be either one or a combination of any two or more physiological and/or biomechanical sensors. The sensors may be, for example, an ECG sensor, accelerometer and gyroscope.

The data may be one or more of the following parameters: heart rate, respiration rate, heart rate variability such as fluctuations of ECG R-R intervals, accelerometer measurements such as accelerometer activity counts, step counts and linear and angular accelerations and gyroscope measurements such as angular speed and/or distance.

The device input may be arranged to receive the data over a wireless connection or over a physical connection.

According to another aspect of the present invention there is provided a method comprising receiving data from a body sensor, estimating energy expenditure using an aerobic metabolism model, determining from the data whether the anaerobic threshold for the user has been exceeded, outputting the estimated energy expenditure if the anaerobic threshold for the user has not been exceeded, estimating energy expenditure due to anaerobic respiration, combining the estimated anaerobic energy expenditure and the estimate aerobic energy expenditure to produce a total energy expenditure when the anaerobic threshold for the user has been exceeded and outputting the total energy expenditure if the anaerobic threshold for the user has been exceeded.

Estimating energy expenditure using an aerobic metabolism model may include classifying the exercise of the user using data received by the input and estimating physical activity intensity and the estimated oxygen level in the respiratory gases according to the classification of the exercise wherein the output outputs the estimated physical activity intensity if the anaerobic threshold for the user has not been exceeded.

Estimating energy expenditure due to anaerobic respiration may include estimating plasma lactate concentration and converting the estimated plasma lactate concentration to a calorie value representing the anaerobic energy. The method also includes calculating a calorie value representing the aerobic energy expenditure using the estimated oxygen level in the respiratory gases and adding the calorie value representing the anaerobic energy and the calorie value representing the aerobic energy expenditure to provide an estimate of the total energy expenditure when the anaerobic threshold has been passed.

The plasma lactate concentration may be estimated using for example an existing method [reference]:

LA=e^{(0.082logVO}^{₂}^{-0.2)} where VO₂<1.51

LA=e^{(2.88logVO}^{₂}^{-0.7)} where VO₂≧1.51

where LA is the plasma lactate concentration.

Converting the estimated plasma lactate concentration to a calorie value representing the anaerobic energy may use the equation:

$${PAI}_{anaerobic} = \frac{20 LA}{0.75}$$

Calculating a calorie value representing the aerobic energy expenditure may use the following equation:

PAI_{aerobic}=5.01*VO₂

The data may be one or more of the following parameters: heart rate, respiration rate, heart rate variability such as fluctuations of ECG R-R intervals, accelerometer measurements such as accelerometer activity counts, step counts and linear and angular accelerations and gyroscope measurements such as angular speed and/or distance.

### BRIEF DESCRIPTION OF THE DRAWINGS

FIG. 1 illustrates a device according to the present invention.

### DETAILED DESCRIPTION

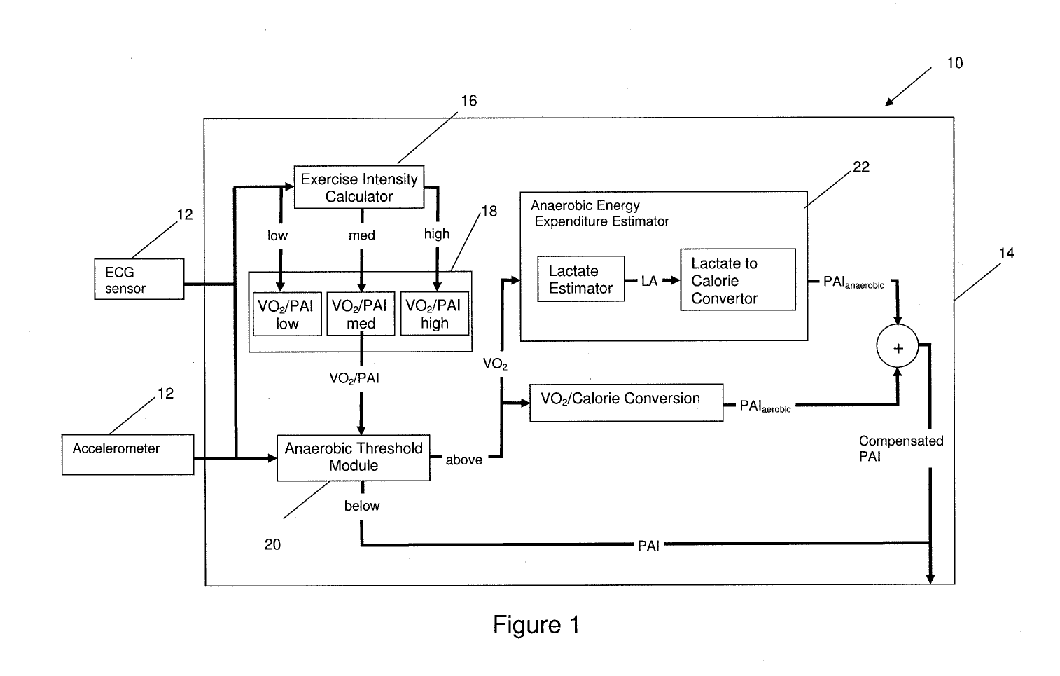

A device 10 for calculating energy expenditure is illustrated in FIG. 1. The device 10 includes sensors 12 such as an ECG sensor and an accelerometer, a processor 14 and a display (not shown).

The ECG sensor and accelerometer are attached to the user's body using any suitable means. The processor 14 receives an input from each of the sensors 12. The sensor input is passed to an exercise intensity classifier 16 which uses the input from one or more of the sensors 12 to classify the current activity intensity as “low”, “moderate” or “high”. For example, the exercise intensity classifier 16 may receive a heart rate and heart rate variability characteristics from an ECG sensor and use these values to calculate the exercise intensity in accordance with any suitable method.

The activity intensity classification is relayed to a Physical Activity Intensity and VO₂ estimator 18 where VO₂ is the estimated oxygen level in the respiratory gases. The Physical Activity Intensity and VO₂ estimator 18 estimates the Physical Activity Intensity and VO₂ of the user using the input from the one or more sensors 12 and relationships between the sensor inputs and the Physical Activity Intensity and VO₂. The relationships between the sensor inputs and the Physical Activity Intensity and VO₂ have been empirically derived using aerobic metabolism models for each of the different activity intensities. Thus, the Physical Activity Intensity value provided by the estimator is a value for the Physical Activity Intensity for aerobic metabolism at a given activity intensity.

Once the physical activity intensity and VO₂ values have been estimated they are passed to an anaerobic threshold module 20. The anaerobic threshold module 20 determines whether the anaerobic threshold for the user of the device has been exceeded or not.

For example, the anaerobic threshold module may determine if the anaerobic threshold has been exceeded based on the extent of variation in the heart rate variability (which may be received from the ECG sensor 12), respiration, as well as accelerometer values. One example of a similar method is described in Michele R D, Gatta G, Leo A D, Cortesi M, Andina F, Tam E, Boit M D, Merni F Estimation of the anaerobic threshold from heart rate variability in an incremental swimming test J Strength Cond Res. Dec. 20, 2011

Alternatively, the anaerobic threshold may be calculated using the age of the user and the heart rate of the user, for example, a maximum heart rate may be given by the equation 220—user age. The anaerobic threshold may then be calculated by calculating a percentage (say 85 %) of this maximum heart rate. The threshold may be stored in a memory in the device and retrieved by the anaerobic threshold module 20. Any other suitable method may be used to estimate the anaerobic threshold.

The heart rate input by the ECG sensor can be compared to the estimated heart rate at the anaerobic threshold. If the heart rate input is greater than the estimated heart rate at the anaerobic threshold the anaerobic threshold module will determine that the anaerobic threshold has been passed. Conversely, if the heart rate input is less than the estimated heart rate at the anaerobic threshold the anaerobic threshold module will determine that the anaerobic threshold has not been passed

If the anaerobic threshold for the user has not been passed then the user is predominantly using aerobic metabolism and therefore the calculated physical activity intensity is an accurate estimate of the actual physical activity intensity and output to the display for the user to view.

If the anaerobic threshold for the user has been passed then the user is predominantly using anaerobic metabolism. In this instance the anaerobic threshold module 20 invokes a lactate estimator 22 to estimate the plasma lactate concentration from the estimated VO₂ value.

The plasma lactate concentration may be estimated using any suitable known relationship between plasma lactate concentration and a physical feature of the user's body. For example, the plasma lactate concentration may be estimated using the VO₂ estimate using the following equations:

LA=e^{(0.082logVO}^{₂}^{-0.2)} where VO₂<1.51

LA=e^{(2.88logVO}^{₂}^{-0.7)} where VO₂≧1.51

where LA is the estimated plasma lactate concentration.

(derived from W. L. Beaver, K. W., and B. J. Whipp, “Improved detection of lactate threshold during exercise using a log-log transformation”. Applied Physiology, 1985. 59: p. 1936-1940)

The estimated plasma lactate concentration is then passed to a lactate to calorie convertor which converts the estimated plasma lactate concentration into calories. One possible method for converting lactate concentration into calories is described in R. Margaria, P. C., F. Mangili, “Balance and kinetics of anaerobic energy release during strenuous exercise in man”. Applied Physiology, 1964. 19: p. 623-628. In this document the relationship between lactate concentration and calories was derived to be:

$${PAI}_{anaerobic} = \frac{20 LA}{0.75}$$

At the same time the estimated VO₂ value is also converted into calories to obtain an estimate of the calories associated with aerobic metabolism. One possible equation for estimating calories using VO₂ concentration is:

PAI_{aerobic}=5.01*VO₂

PAI_{anaerobic} is combined with PAI_{aerobic} to give a final compensated energy expenditure value which can be output to display.

In this way the user can be provided with an accurate estimate of energy expenditure when they are exercising beyond their aerobic capacity.

As will be understood by the skilled person the Physical Activity Intensity and VO₂ values provided by the Physical Activity Intensity and VO₂ estimator may be calculated using any suitable method provided the VO₂ and PAI are estimated as if the anaerobic threshold has not been breached i.e. assuming that the rate of increase in carbon dioxide is proportional to the increase in oxygen uptake. For example, the method described in US 2008/275348 may be used.

Although the present invention has been described with the Physical Activity Intensity and VO₂ values provided by the Physical Activity Intensity and VO₂ estimator being passed to an anaerobic threshold module the skilled person will understand that the anaerobic threshold module may use the input of the sensors to determine whether the anaerobic threshold has been passed concurrently with the Exercise Intensity and/or Physical Activity Intensity and VO₂ values being estimated. The output of the anaerobic threshold module may then be used to determine whether to output the Physical Activity Intensity and VO₂ values or pass the Physical Activity Intensity and VO₂ values to the anaerobic energy expenditure estimator with the anaerobic threshold module never receiving the Physical Activity Intensity and VO₂ values.

Alternatively, the anaerobic threshold module may use the input of the sensors to determine whether the anaerobic threshold has been passed before the Exercise Intensity and/or Physical Activity Intensity and VO₂ values being estimated. The output of the anaerobic threshold module may then be used to determine whether to use the aerobic model to estimate the Physical Activity Intensity and VO₂ values or pass the Physical Activity Intensity and VO₂ values to the anaerobic energy expenditure estimator with the anaerobic threshold module never receiving the Physical Activity Intensity and VO₂ values.

The skilled person will also understand that, although the present invention, describes the measurement of energy being calories, any suitable unit of energy may be calculated and output to the user.

The sensors may be any suitable sensor for monitoring characteristics of the user's body. For example, the sensor may be one or more of a heart rate monitor, an accelerometer and a gyroscope. The sensor may transmit recorded data using any suitable means. For example, the sensor may transmit the data wirelessly or through a wire connected to the device.

Additionally, the device may not only be provided with a display but additionally, or alternatively may be provided with any suitable means to output the calculated energy expenditure to a separate device. The device may be, for example, a personal computer, a remote server or any other suitable device.

Additionally, the device may be integrated into other devices. For example, it may form part of a user's cellular telephone.

1. A device comprising:
an input configured to receive data from a sensor;
an aerobic processor configured to estimate aerobic energy expenditure from the data using an aerobic metabolism model;
an anaerobic threshold module configured to determine from the data whether the anaerobic threshold for the user has been exceeded;
an output to output the estimated physical activity intensity if the anaerobic threshold for the user has not been exceeded;
an anaerobic energy expenditure estimator configured to estimate anaerobic energy expenditure due to anaerobic respiration from the data;
a processor configured to combine the estimated anaerobic energy expenditure and the estimate aerobic energy expenditure to produce a total energy expenditure when the anaerobic threshold for the user has been exceeded;
an output to output the total energy expenditure if the anaerobic threshold for the user has been exceeded.

2. A device as claimed in claim 1 wherein the aerobic processor comprises:
an exercise intensity classifier configured to classify the exercise of the user using data received by the input;
an estimator to estimate physical activity intensity and the estimated oxygen level in the respiratory gases according to the classification of the exercise wherein the output outputs the estimated physical activity intensity if the anaerobic threshold for the user has not been exceeded.

3. A device as claimed in claim 1 wherein the anaerobic energy expenditure estimator comprises:
a lactate estimator to estimate plasma lactate concentration; and
a lactate to calorie convertor to convert the estimated plasma lactate concentration to a calorie value representing the anaerobic energy;
and the device further comprises:
an aerobic energy expenditure estimator configured to calculate a value representing the aerobic energy expenditure using the estimated oxygen level in the respiratory gases;
a processor configured to add the calorie value representing the anaerobic energy and the calorie value representing the aerobic energy expenditure to provide an estimate of the total energy expenditure when the anaerobic threshold has been passed.

4. A device as claimed in claim 3 wherein the plasma lactate concentration estimator estimates plasma lactate concentration using:
LA=e^{(0.082logVO}^{₂}^{-0.2)} where VO₂<1.51
LA=e^{(2.88logVO}^{₂}^{-0.7)} where VO₂≧1.51
where LA is the plasma lactate concentration

5. A device as claimed in claim 4 wherein the estimated plasma lactate concentration is converted to a calorie value representing the anaerobic energy using the equation:
$${PAI}_{anaerobic} = \frac{20 LA}{0.75}$$

6. A device as claimed in claim 4 wherein the anaerobic energy expenditure estimator calculates the calories using the following equation:
PAI_{aerobic}=4.76*VO₂

7. A device as claimed in claim 1 wherein the body sensor comprises one or more of an ECG, an accelerometer and a gyroscope.

8. A device as claimed in claim 1 wherein the data comprises one or more of heart rate, respiration rate, heart rate variability, fluctuations in the ECG R-R intervals, accelerometer measurements and gyroscope measurements.

9. A device as claimed in claim 1 wherein the input is configured to receive the data over a wireless connection.

10. A method comprising
receiving data from a body sensor;
estimating energy expenditure using an aerobic metabolism model;
determining from the data whether the anaerobic threshold for the user has been exceeded;
outputting the estimated energy expenditure if the anaerobic threshold for the user has not been exceeded;
estimating energy expenditure due to anaerobic respiration;
combining the estimated anaerobic energy expenditure and the estimate aerobic energy expenditure to produce a total energy expenditure when the anaerobic threshold for the user has been exceeded; and
outputting the total energy expenditure if the anaerobic threshold for the user has been exceeded.

11. A method as claimed in claim 10 wherein estimating energy expenditure using an aerobic metabolism model comprises:
classifying the exercise of the user using data received by the input; and
estimating physical activity intensity and the estimated oxygen level in the respiratory gases according to the classification of the exercise wherein the output outputs the estimated physical activity intensity if the anaerobic threshold for the user has not been exceeded.

12. A method as claimed in claim 10 wherein estimating energy expenditure due to anaerobic respiration comprises:
estimating plasma lactate concentration; and
converting the estimated plasma lactate concentration to a calorie value representing the anaerobic energy
and the method further comprises
calculating a calorie value representing the aerobic energy expenditure using the estimated oxygen level in the respiratory gases; and
adding the calorie value representing the anaerobic energy and the calorie value representing the aerobic energy expenditure to provide an estimate of the total energy expenditure when the anaerobic threshold has been passed.

13. A method as claimed in claim 12 wherein the plasma lactate concentration is estimated using:
LA=e^{(0.082logVO}^{₂}^{-0.2)} where VO₂<1.51
LA=e^{(2.88logVO}^{₂}^{-0.7)} where VO₂≧1.51
where LA is the plasma lactate concentration.

14. A method as claimed in claim 12 wherein converting the estimated plasma lactate concentration to a calorie value representing the anaerobic energy is performed using the equation:
$${PAI}_{anaerobic} = \frac{20 LA}{0.75}$$

15. A method as claimed in claim 12 wherein calculating a calorie value representing the aerobic energy expenditure uses the following equation:
PAI_{aerobic}=4.76*VO₂

16. A method as claimed in claim 10 wherein the data comprises one or more of heart rate, variability in the ECG R-R intervals, respiratory rate, accelerometer measurements and gyroscope measurements.
